(19) United States
(12) Patent Application Publication
(10) Pub. No.: US 2023/0011611 A1
(43) Pub. Date: Jan. 12, 2023

(54) SURGICAL INSTRUMENT WITH AUTOMATED ACTIVATION

(71) Applicant: Covidien LP, Mansfield, MA (US)

(72) Inventor: Zachary S. Heiliger, Nederland, CO (US)

(21) Appl. No.: 17/858,355

(22) Filed: Jul. 6, 2022

Related U.S. Application Data
(60) Provisional application No. 63/218,616, filed on Jul. 6, 2021.

(51) Int. Cl. A61B 18/14; A61B 18/12

(52) U.S. Cl. CPC A61B 18/1445; A61B 18/1206; A61B 2018/00708

(57) ABSTRACT

A surgical instrument includes a housing having a shaft extending therefrom for supporting an end effector assembly. A handle is disposed on the housing and is selectively moveable relative thereto to actuate the end effector assembly. A PCB is disposed within the housing and includes one or more accelerometers and a timing circuit having a first timer. The accelerometer is configured to activate the first timer of the timing circuit upon detecting movement of the surgical instrument after the surgical instrument is coupled to an electrosurgical energy source. A deactivating assembly is disposed within the housing and is operably associated with the timing circuit such that after expiration of the first timer, the deactivation assembly mechanically decommissions the surgical instrument for continued or subsequent use.

### CROSS REFERENCE TO RELATED APPLICATION

This application claims the benefit of U.S. Provisional Application Ser. No. 63/218,616 filed Jul. 6, 2021, the entire contents of which being incorporated by reference herein.

### BACKGROUND

### 1. Technical Field

The present disclosure relates generally to the field of surgical instruments. In particular, the disclosure relates to an endoscopic electrosurgical forceps that is configured to automatically initialize with a generator after electrical communication therewith and upon subsequent movement thereof.

### 2. Background of Related Art

Instruments such as electrosurgical forceps are commonly used in open and endoscopic surgical procedures to coagulate, cauterize and seal tissue. Such forceps typically include a pair of jaw members that can be controlled by a surgeon to grasp targeted tissue, such as, e.g., a blood vessel. The jaw members may be approximated to apply a mechanical clamping force to the tissue, and are associated with at least one electrode to permit the delivery of electrosurgical energy to the tissue. The combination of the mechanical clamping force and the electrosurgical energy has been demonstrated to join adjacent layers of tissue captured between the jaw members. When the adjacent layers of tissue include the walls of a blood vessel, sealing the tissue may result in hemostasis, which may facilitate the transection of the sealed tissue. A detailed discussion of the use of an electrosurgical forceps may be found in U.S. Pat. No. 7,255,697 to Dycus et al.

A bipolar electrosurgical forceps typically includes opposed electrodes disposed on clamping faces of the jaw members. The electrodes are charged to opposite electrical potentials such that an electrosurgical current may be selectively transferred through tissue grasped between the electrodes. To effect a proper seal, particularly in relatively large vessels, two predominant mechanical parameters must be accurately controlled; the pressure applied to the vessel, and the gap distance established between the electrodes.

Both the pressure and gap distance influence the effectiveness of the resultant tissue seal. If an adequate gap distance is not maintained, there is a possibility that the opposed electrodes will contact one another, which may cause a short circuit and prevent energy from being transferred through the tissue. Also, if too low a force is applied the tissue may have a tendency to move before an adequate seal can be generated. The thickness of a typical effective tissue seal is optimally between about 0.001 and about 0.006 inches. Below this range, the seal may shred or tear and above this range the vessel walls may not be effectively joined. Closure pressures for sealing large tissue structures preferably fall within the range of about 3 kg/cm² to about 16 kg/cm².

Prior to surgical use, a surgeon or a surgical prep team typically plugs the various electrosurgical instruments into one or more generators in anticipation for use during surgery. As a result thereof, the instruments are electrically live and typically initialized and registered with the generator prior to use and before introduction into the surgical field. In some cases, there may be a prolonged period between initialization and actual use.

### SUMMARY

The present disclosure relates to an electrosurgical apparatus and methods for performing electrosurgical procedures. More particularly, the present disclosure relates to electrosurgically sealing tissue.

Providence herein in accordance with the present disclosure is a surgical instrument that includes a housing having a shaft extending therefrom. An end effector assembly is disposed at a distal end of the shaft and includes first and second jaw members movable between a first position wherein at least one of the jaw members is spaced relative to the other of the jaw members and a second position wherein the first and second jaw members cooperate to grasp tissue. One or both of the jaw members is adapted to connect to an electrosurgical energy source. A handle is disposed on the housing and is selectively moveable relative thereto to move the jaw members between the first and second positions.

A PCB is disposed within the housing and includes one or more accelerometers and a timing circuit having a first timer. The at least one accelerometer is configured to activate the first timer of the timing circuit upon detecting movement of the surgical instrument after the surgical instrument is coupled to the electrosurgical energy source. A deactivating assembly is disposed within the housing and is operably associated with the timing circuit such that after expiration of the first timer, the deactivation assembly mechanically decommissions the surgical instrument for continued or subsequent use.

In aspects according to the present disclosure, the deactivating assembly mechanically decommissions the surgical instrument for continued use by severing internal electrical connections to the jaw members. In other aspects according to the present disclosure, the deactivating assembly includes a blade that selectively extends to sever one or more wire conductors connected to one or both jaw members upon expiration of the first timer. In still other aspects according to the present disclosure, the deactivating assembly includes an actuator operably coupled to the blade, the actuator configured to selectively extend the blade upon expiration of the first timer. In yet other aspects according to the present disclosure, the actuator includes at least one of a spring or solenoid.

In aspects according to the present disclosure, the PCB includes a 3-axis accelerometer disposed thereon which is configured to detect handling of the surgical instrument. In other aspects according to the present disclosure, the PCB board includes vibration sensor configured to sense vibration of the surgical instrument during handling.

Providence herein in accordance with other aspects of the present disclosure is a surgical instrument that includes a housing having a shaft extending therefrom that supports an end effector assembly. A PCB is disposed within the housing and includes one or more accelerometers and a timing circuit having a first timer. The one or more accelerometers is configured to activate the first timer of the timing circuit upon detecting movement of the surgical instrument after the surgical instrument is coupled to an electrosurgical energy source. A deactivating assembly is disposed within the housing and is operably associated with the timing circuit such that after expiration of the first timer, the deactivation assembly mechanically decommissions the surgical instrument for continued or subsequent use.

In aspects according to the present disclosure, the deactivating assembly mechanically decommissions the surgical instrument for continued use by severing internal electrical connections to the end effector assembly. In other aspects according to the present disclosure, the deactivating assembly includes a blade that selectively extends to sever one or more wire conductors connected to the end effector assembly upon expiration of the first timer. In still other aspects according to the present disclosure, the deactivating assembly includes an actuator operably coupled to the blade, the actuator configured to selectively extend the blade upon expiration of the first timer. In yet other aspects according to the present disclosure, the actuator includes at least one of a spring or solenoid.

In aspects according to the present disclosure, the PCB includes a 3-axis accelerometer disposed thereon which is configured to detect handling of the surgical instrument. In other aspects according to the present disclosure, the PCB board includes vibration sensor configured to sense vibration of the surgical instrument during handling.

Providence herein in accordance with other aspects of the present disclosure is a method of decommissioning an electrosurgical instrument and includes: coupling an electrosurgical instrument having an end effector assembly to an electrosurgical energy source; using the electrosurgical instrument during a surgical procedure to induce an accelerometer to activate a first timer of a timer circuit of a PCB; and mechanically deactivating the electrosurgical instrument after expiration of the first timer to decommission the electrosurgical instrument for continued or subsequent use.

In aspects according to the present disclosure, mechanically deactivating the electrosurgical instrument after expiration of the first timer includes severing internal electrical connections to the end effector assembly using a deactivating assembly. In other aspects according to the present disclosure, the deactivating assembly includes a blade and an actuator operably coupled to the blade, the actuator configured to selectively extend the blade upon expiration of the first timer.

In aspects according to the present disclosure, the accelerometer is a 3-axis accelerometer.

In aspects according to the present disclosure, the method includes initializing the electrosurgical instrument after coupling the electrosurgical instrument to the electrosurgical energy source. In other aspects according to the present disclosure, the first timer is activated only after initialization of the electrosurgical instrument.

### BRIEF DESCRIPTION OF THE DRAWINGS

The accompanying drawings, which are incorporated in and constitute a part of this specification, illustrate embodiments of the present disclosure and, together with the detailed description of the embodiments given below, serve to explain the principles of the disclosure.

FIG. 1 is a perspective view of an electrosurgical forceps according to an embodiment of the present disclosure including a housing, an elongated shaft, and an end effector;

FIG. 2A is an enlarged, perspective view of the end effector of FIG. 1 depicted with a pair of jaw members in an open configuration;

FIG. 2B is an enlarged, perspective view of the end effector of FIG. 1 depicted with the pair of jaw members in a closed configuration;

FIG. 3A is a perspective view of the end effector and elongated shaft of FIG. 1 with parts separated;

FIG. 3B is cross-sectional view taken along line 3B-3B of FIG. 3A showing a distal portion of the electrosurgical forceps of FIG. 1 depicting a tube guide;

FIG. 4 is a perspective view of a proximal portion of the instrument of FIG. 1 with a portion of the housing removed revealing internal components;

FIG. 5A is a side view of an electrosurgical forceps according to another embodiment of the present disclosure including a housing, an elongated shaft, an end effector assembly, PCB and deactivating assembly;

FIG. 5B is an embodiment of the PCB board of FIG. 5A; and

FIG. 5C is an embodiment of the deactivating assembly of FIG. 5A.

FIG. 6 is a schematic diagram of a timing circuit for use with the present disclosure.

### DETAILED DESCRIPTION

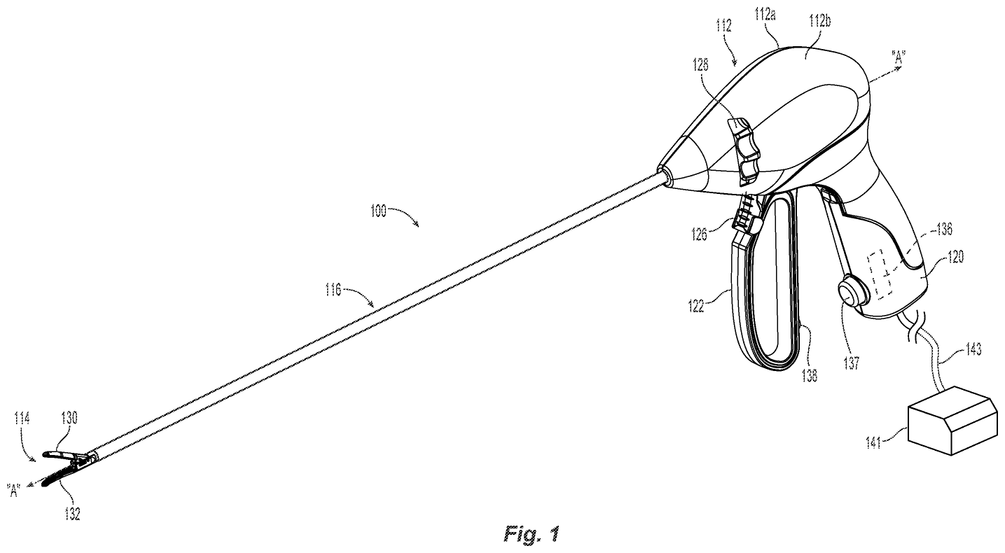

Referring initially to FIG. 1, an electrosurgical forceps 100 generally includes a housing 112 that supports various actuators thereon for remotely controlling an end effector 114 through an elongated shaft 116. Although discussed herein as electrosurgical forceps 100, it is envisioned that any electrosurgical instrument may be utilized with the presently disclosure embodiments here. Moreover and although this configuration is typically associated with instruments for use in laparoscopic or endoscopic surgical procedures, various aspects of the present disclosure may be practiced with traditional open instruments and in connection with endoluminal procedures as well. The housing 112 is constructed of a left housing half 112a and a right housing half 112b. The left and right designation of the housing halves 112a, 112b refer to the respective directions as perceived by an operator using the forceps 100. The housing halves 112a, 112b may be constructed of sturdy plastic, and may be joined to one another by adhesives, ultrasonic welding or other suitable assembly methods.

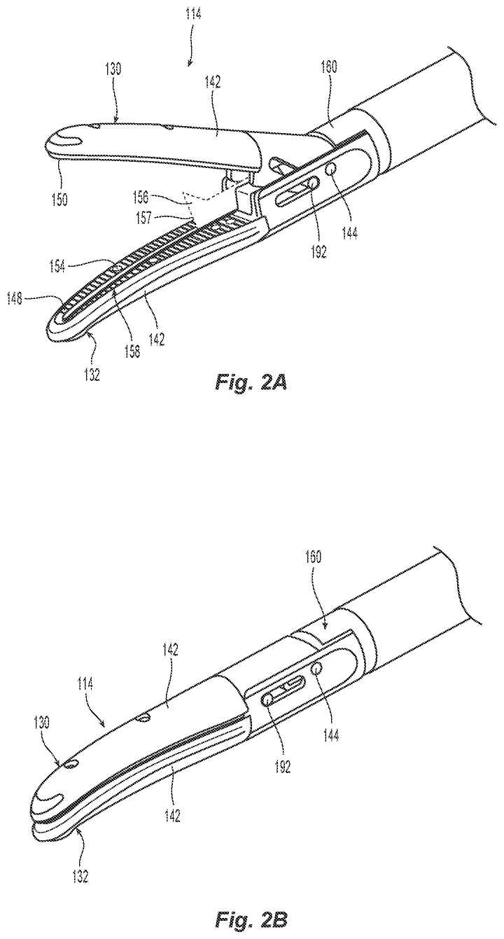

To mechanically control the end effector 114, the housing 112 supports a stationary handle 120, a movable handle 122, a trigger 126 and a rotation knob 128. The movable handle 122 is operable to move the end effector 114 between an open configuration (FIG. 2A) wherein a pair of opposed jaw members 130, 132 are disposed in spaced relation relative to one another, and a closed or clamping configuration (FIG. 2B) wherein the jaw members 130, 132 are closer together. Approximation of the movable handle 122 with the stationary handle 120 serves to move the end effector 114 to the closed configuration and separation of the movable handle 122 from the stationary handle 120 serves to move the end effector 114 to the open configuration. The trigger 126 is operable to extend and retract a knife blade 156 (see FIGS. 2A and 2B) through the end effector 114 when the end effector 114 is in the closed configuration. The rotation knob 128 serves to rotate the elongated shaft 116 and the end effector 114 about a longitudinal axis A-A extending through the forceps 114.

To electrically control the end effector 114, the stationary handle 120 supports a depressible button 137 thereon, which is operable by the user to initiate and terminate the delivery of electrosurgical energy to the end effector 114. The depressible button 137 is mechanically coupled to a switch 136 disposed within the stationary handle 120 and is engageable by a button activation post 138 extending from a proximal side of the moveable handle 122 upon proximal movement of the moveable handle 122 to an actuated or proximal position. The switch 136 is in electrical communication with an electrosurgical generator 141 via suitable electrical wiring 143a, 143b extending from the housing 112 through a cable 143 extending between the housing 112 and the electrosurgical generator 141. The generator 141 may include devices such as the LigaSure® Vessel Sealing Generator and the ForceTriad® Generator sold by Covidien. The cable 143 may include a connector (not shown) thereon such that the forceps 100 may be selectively coupled electrically to the generator 141.

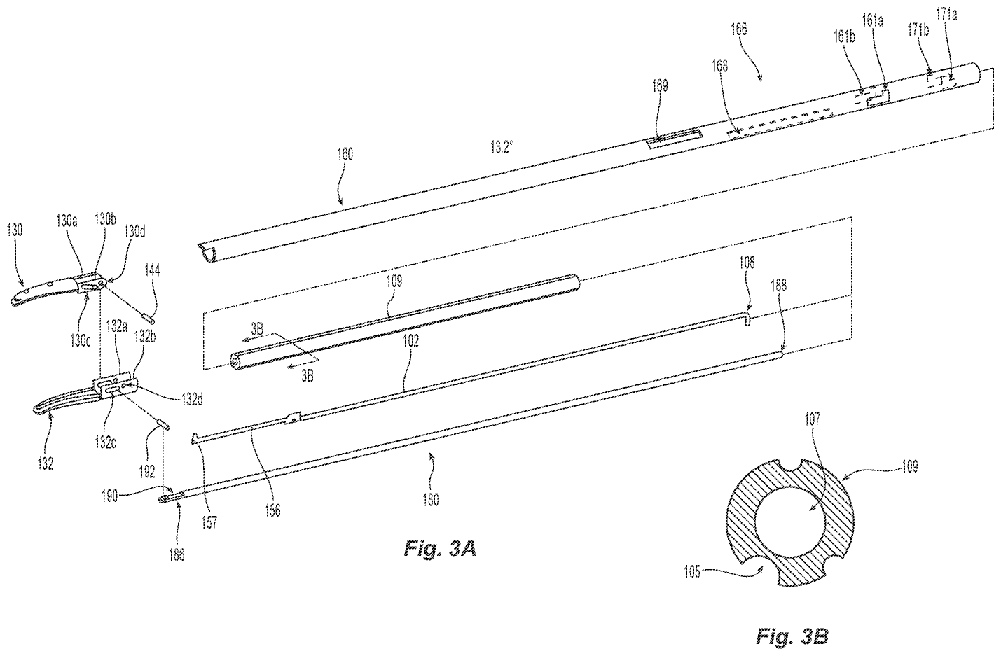

Referring now to FIGS. 2A-3A, the end effector 114 may be moved from the open configuration (FIG. 2A) wherein tissue (not shown) is received between the jaw members 130, 132, and the closed configuration (FIG. 2B), wherein the tissue is clamped and treated. The jaw members 130, 132 pivot about a pivot pin 144 to move the end effector 114 to the closed configuration of FIG. 2B wherein the sealing plates 148, 150 provide a pressure to tissue grasped therebetween. In some embodiments, to provide an effective tissue seal, a pressure within a range between about 3 kg/cm² to about 16 kg/cm² and, desirably, within a working range of about 7 kg/cm² to about 13 kg/cm², may be applied to the tissue.

Also, in the closed configuration, a separation or gap distance is maintained between the sealing plates 148, 150 by an array of stop members 154 (FIG. 2A) disposed on or adjacent the sealing plates 148, 150. The stop members 154 contact opposing surfaces on the opposing jaw member 130, 132 and prohibit further approximation of the sealing plates 148, 150. In some embodiments, to provide an effective tissue seal, an appropriate gap distance of about 0.001 inches to about 0.010 inches and, desirably, between about 0.002 inches to about 0.005 inches, may be provided. In some embodiments, the stop members 154 are constructed of a heat-resistant ceramic deposited onto the jaw members 130, 132. In other embodiments, the stop members 154 are constructed of an electrically non-conductive plastic molded onto the jaw members 130, 132, e.g., by a process such as overmolding or injection molding.

The upper and lower jaw members 130, 132 are electrically coupled to cable 143, and thus to the generator 141 (e.g., via respective suitable electrical wiring extending through the elongated shaft 116) to provide an electrical pathway to a pair of electrically conductive, tissue-engaging sealing plates 148, 150 disposed on the lower and upper jaw members 132, 130, respectively. The sealing plate 148 of the lower jaw member 132 opposes the sealing plate 150 of the upper jaw member 130. In some embodiments, the sealing plates 148 and 150 are electrically coupled to opposite terminals, e.g., positive or active (+) and negative or return (—) terminals associated with the generator 141. Thus, bipolar energy may be provided through the sealing plates 148 and 150 to tissue. Alternatively, the sealing plates 148 and 150 may be configured to deliver monopolar energy to tissue. In a monopolar configuration, one or both sealing plates 148 and 150 deliver electrosurgical energy from an active terminal, e.g., (+), while a return pad (not shown) is placed generally on a patient and provides a return path to the opposite terminal, e.g., (−), of the generator 141. Each jaw member 130, 132 includes a jaw insert 140 and an insulator 142 that serves to electrically insulate the sealing plates 150, 148 from the jaw insert 140 of the jaw members 130, 132, respectively.

Electrosurgical energy may be delivered to the tissue through the electrically conductive seal plates 148, 150 to effect a tissue seal. Once a tissue seal is established, a knife blade 156 having a sharpened distal edge 157 may be advanced through a knife channel 158 defined in one or both jaw members 130, 132 to transect the sealed tissue. Although the knife blade 156 is depicted in FIG. 2A as extending from the elongated shaft 116 when the end effector 114 is in an open configuration, in some embodiments, extension of the knife blade 156 into the knife channel 158 when the end effector 114 is in the open configuration is prevented.

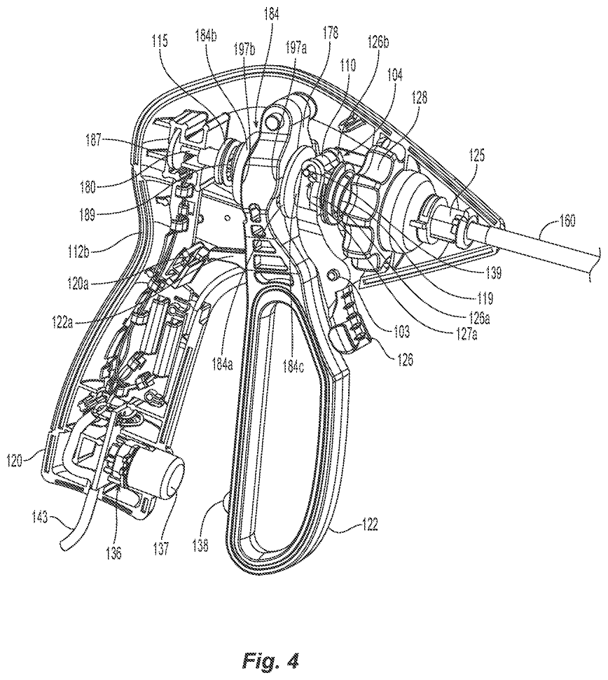

Referring to FIG. 3A, the elongated shaft 116 includes various longitudinal components that operatively couple the end effector 114 to the various actuators supported by the housing 112 (FIG. 1). An outer shaft member 160 defines an exterior surface of the elongated shaft 116 and houses other components therein as described below. The outer shaft member 160 is configured for longitudinal motion with respect to an inner actuation member 180 axially received within the outer shaft member 160. The inner actuation member 180 may be a rod, a shaft, a tube, folded metal, stamped metal, or other suitable structure. A proximal portion 166 of the outer shaft member 160 is configured for receipt within the housing 112 (FIG. 1), and includes features for operatively coupling the outer shaft member 160 to various elements of the housing 112. More specifically, the proximal portion 166 of the outer shaft member 160 includes, in order from distal to proximal, a longitudinal slot 169 to couple the outer shaft member 160 to the rotation knob 128, a longitudinal knife slot 168 defined therethrough, a pair of opposing distal locking slots 161a, 161b, and a pair of opposing proximal locking slots 171a, 171b. The connection established between the outer shaft member 160 and the rotation knob 128 is described below with reference to FIG. 4.

A distal portion 186 of the inner actuation member 180 includes a longitudinal recess 190 defined therein that provides clearance for the pivot pin 144 and thus, permits longitudinal reciprocation of the pivot pin 144 (via longitudinal reciprocation of the outer shaft member 160) independent of the inner actuation member 180. Distally of the longitudinal recess 190, a cam pin 192 is mechanically coupled (e.g., via welding, friction-fit, laser welding, etc) to the distal portion 186 of the inner actuation member 180. A proximal portion 188 of the inner actuation member 180 includes a washer 187 coupled thereto (FIG. 4). The washer 187 is captured within the housing 112 and serves to prohibit longitudinal motion of the inner actuation member 180 parallel to the longitudinal axis A-A.

The pivot pin 144 extends through a proximal portion of each of the jaw members 130, 132 to pivotally support the jaw members 130, 132 at the distal end of the inner actuation member 180. A proximal portion of each of the jaw members 130, 132 includes two laterally spaced parallel flanges or “flags” 130a, 130b and 132a, 132b respectively, extending proximally from a distal portion of the jaw members 130 and 132 (FIG. 3A). A lateral cam slot 130c and a lateral pivot bore 130d extend through each of the flags 130a, 130b of the upper jaw member 130 (FIG. 3A). Similarly, a lateral cam slot 132c and a lateral pivot bore 132d extend through each of the flags 132a, 132b of the lower jaw member 132. The pivot bores 130d, 132d receive the pivot pin 144 in a slip-fit relation that permits the jaw members 130, 132 to pivot about the pivot pin 144 to move the end effector 114 between the open and closed configurations (FIGS. 2A and 2B, respectively).

A knife rod 102 is coupled (e.g., via welding) at a distal-most end to the sharpened knife blade 156 and includes an angled proximal end 108 that provides a mechanism for operatively coupling the knife rod 102 to the trigger 126. In some embodiments, the angled proximal end 108 of the knife rod 102 is formed by bending the knife rod 102 ninety degrees at its proximal end during manufacturing. The connection between the knife rod 102 and the trigger 126 is described in detail below with reference to FIG. 4. The sharpened distal edge 157 of the knife blade 156 may be applied to the distal end of the knife blade 156 using a variety of manufacturing techniques such as, for example, grinding, coining, electrochemical etching, electropolishing, or other suitable manufacturing technique, for forming sharpened edges. Alternatively, an electrical cutter may be positioned between jaw members 130, 132 to sever tissue disposed therebetween upon activation thereof.

Referring to FIGS. 3A and 3B, a tube guide 109 is disposed within the outer shaft member 160 and includes a lumen 107 axially disposed therethrough. The inner actuation member 180 is received within the guide lumen 107, which serves to orient and align the inner actuation member 180 within the outer shaft member 160. The knife rod 102 is received within a longitudinal guide recess 105 formed in the outer surface of the guide tube 109. The guide recess 105 serves to guide longitudinal motion of the knife rod 102 within the outer shaft member 160 and to radially space the knife rod 102 from the inner actuation member 180 to prevent the inner actuation member 180 from interfering with reciprocal motion of the knife rod 102.

Referring now to FIG. 4, the connection of the movable handle 122 and the knife trigger 126 to the longitudinally movable components of the elongated shaft 116 is described. The movable handle 122 may be manipulated to impart longitudinal motion to the outer shaft member 160, and the knife trigger 126 may be manipulated to impart longitudinal motion to the knife rod 102. As discussed above, longitudinal motion of the outer shaft member 160 serves to move the end effector 114 between the open configuration of FIG. 2A and the closed configuration of FIG. 2B, and longitudinal motion of the knife rod 102 serves to move knife blade 156 through knife channel 158 (FIG. 2A).

The movable handle 122 is operatively coupled to the outer shaft member 160 by a clevis 178 defined at an upper end of the movable handle 122. The clevis 178 is pivotally supported on the housing 112. The clevis 178 extends upwardly about opposing sides of a drive collar 184 supported on the outer shaft member 160 and includes rounded drive surfaces 197a and 197b thereon. Drive surface 197a engages a proximal-facing surface of a distal spring washer 184a and drive surface 197b engages a distal facing surface of a proximal rim 184b of the drive collar 184. The distal spring washer 184a engages a proximal facing surface of a distal spring stop 184c that, in turn, engages the opposing distal locking slots 161a, 161b (FIG. 3A) extending through the proximal portion 166 (FIG. 3A) of the outer shaft member 160 to couple the distal spring stop 184c to the outer shaft member 160. The drive surfaces 197a, 197b are arranged along the longitudinal axis A-A such that pivotal motion of the movable handle 122 induces corresponding longitudinal motion of the drive collar 184 along the longitudinal axis A-A.

Distal longitudinal motion is imparted to the outer shaft member 160 by driving the drive collar 184 distally with the movable handle 122. Distal longitudinal motion of the drive collar 184 induces a corresponding distal motion of the outer shaft member 160 by virtue of the coupling of the drive collar 184 to opposing distal locking slots 181a, 181b extending through the proximal portion 166 of the outer shaft member 160 (FIG. 3A).

Proximal longitudinal motion of the outer shaft member 160 draws jaw member 132 proximally such that the cam pin 192 advances distally to pivot jaw member 130 toward jaw member 132 to move the end effector 114 to the closed configuration. Once the jaw members 130 and 132 are closed, the outer shaft member 160 essentially bottoms out (i.e., further proximal movement of the outer shaft member 160 is prohibited since the jaw members 130, 132 contact one another). Further proximal movement of the movable handle 122 (FIG. 4), however, will continue to move the drive collar 184 proximally. This continued proximal movement of the drive collar 184 further compresses the spring 189 to impart additional force to the outer shaft member 160, which results in additional closure force applied to tissue grasped between the jaw members 130, 132 (see FIG. 2B).

Referring again to FIG. 4, the trigger 126 is pivotally supported in the housing 112 about a pivot boss 103 protruding from the trigger 126. The trigger 126 is operatively coupled to the knife rod 102 by a knife connection mechanism 104 such that pivotal motion of the trigger 126 induces longitudinal motion of the knife rod 102. The knife connection mechanism 104 includes upper flanges 126a, 126b of the trigger 126 and a knife collar 110.

The upper flanges 126a, 126b of the trigger 126 include respective slots 127a, 127b defined therethrough that are configured to receive the pin bosses 139a, 139b, respectively, of the knife collar 110 such that pivotal motion of the trigger 126 induces longitudinal motion of the knife collar 110 and, thus, the knife rod 102 by virtue of the coupling of knife rod 102 to the knife collar 110.

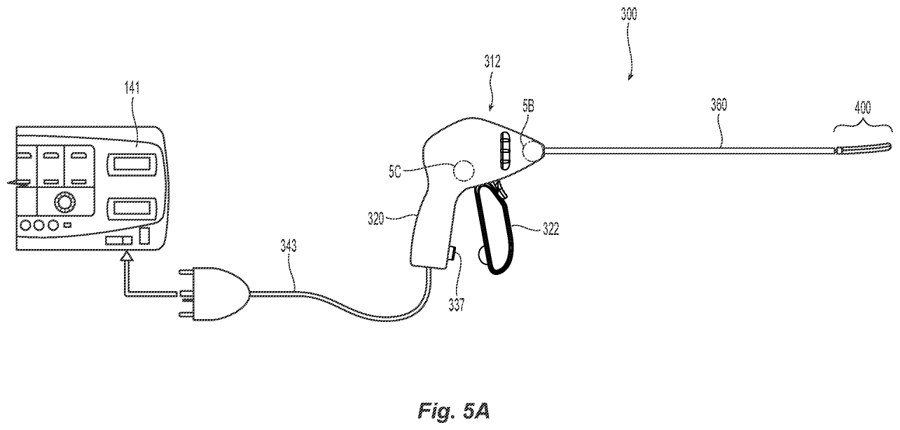

FIGS. 5A-5C show another embodiment of an electrosurgical forceps 300 configured for automated activation in accordance with the present disclosure. Although an electrosurgical forceps 300 is shown, it is envisioned that a non-electrosurgical forceps may be used I accordance with the present disclosure as described herein.

Forceps 300 is similar to the above-described forceps 100 and, as such, only those components that are different are described hereinbelow. Forceps 300 includes housing 312 having a stationary a handle 320 and a movable handle 322 that cooperate to actuated end effector assembly 400 in a similar manner as described above. A shaft 360 extends from housing 312 and supports end effector 400 at a distal end thereof. Depressible button 337 is disposed on stationary handle 320 to allow in-line activation of electrosurgical energy when engaged via actuation of handle 322. A cable 343 including wire conductors 343a, 343b extends from stationary handle 320 and connects to the electrosurgical generator 141.

A PCB 375 having a series of accelerometers is disposed within housing 312 and is configured to electrically communicate with the generator 141 to automatically configure the forceps 300 for activation via depressible button 337 or to initiate a timing circuit 379 as explained in more detail below. More particularly, PCB 375 may include a 3-axis accelerometer 376 which detects movement in three directions (x-axis, y-axis, and z-axis) or three (3) accelerometers (not shown) disposed thereon which each detect movement in a given direction, e.g., x-axis, y-axis, and z-axis. Once movement or handling of the forceps 300 is detected and communicated to the generator 141, the generator is configured to permit activation via depressible button 337. A battery 377 may be included to power the 3-axis accelerometer (s) 376. The PCB 375 may also include a vibration sensor 378 disposed thereon configured to detect vibrational movement of the forceps 300 and communicate handling to the generator 141.

In one embodiment, upon initial use, the forceps 300 is plugged into the generator 141, recognized and initialized for usage. The forceps 300 may then be placed into a first standby mode wherein the forceps 300 is electrically inactive. When the forceps 300 is picked up or otherwise handled, the vibration senor 378 and/or the 3-axis accelerometer 376 detects movement of the forceps 300 and places the forceps 300 in an active mode, e.g., enables electrosurgical activation of the end effector assembly 400 as described above. If the forceps 300 is put aside or placed away from the operating environment, e.g., no longer in use, the forceps 300 is again automatically placed into a second or subsequent standby mode. If the forceps 300 is picked up or handled again, the forceps 300 is placed again into an active mode. A timing circuit 379 may be utilized for this purpose, e.g., the forceps 300 has been movement free for 5 minutes will initiate the second standby mode. Any time frame may be utilized for this purpose, e.g., in the range of 1 minute to 60 minutes or longer.

The first standby mode may be different than the second standby mode and may serve different purposes. For example, upon initial activation and after the forceps 300 is placed into the active mode for the first time, the timing circuit 379 may initiate a first timer that measures the amount of time from the first handling or first use of the forceps 300. It is important to note that the first timer is initiated only after the first handling or use as detected by the vibration sensor 378 and/or the 3-axis accelerometer 376 and not from initialization by the generator 141. In other instances, the first timer may only be initiated after initialization of the forceps 300, e.g., initial recognition by the generator 141 and/or downloading of operating parameters and/or energy controls, etc.

As can be appreciated, prior art devices may be configured to initiate a timer from initialization or when the forceps 300 is initially plugged into the generator 141. The presently disclosed timing circuit 379 only initiates the first timer when first use or first handling is determined which, in some instances can be significantly shorter that first initialization.

The first timer may be a safety measure that counts down a clock that electrically decouples or decommissions the forceps 300 from communicating with the generator 141 after a preset duration, e.g., 2 hours, 4 hours, 6 hours, etc. As can be appreciated, this prevents reuse of the same forceps 300 after surgery and before sterilization (if reusable) or prevents reuse if the forceps 300 is disposable. If reusable, the sterilization process (not described herein) may include one or more steps to recalibrate the first timer.

The second timer of the timing circuit 379 is configured to automatically deactivate the forceps 300 after a preset time (as described above) and place the forceps 300 in a second standby mode and reactivate the forceps 300 when again handled. As can be appreciated, placing the forceps 300 in an inactive mode when not being used enhances OR safety. Moreover, the automated re-activation of the forceps 300 when re-handled does not place any additional burden on operating personnel.

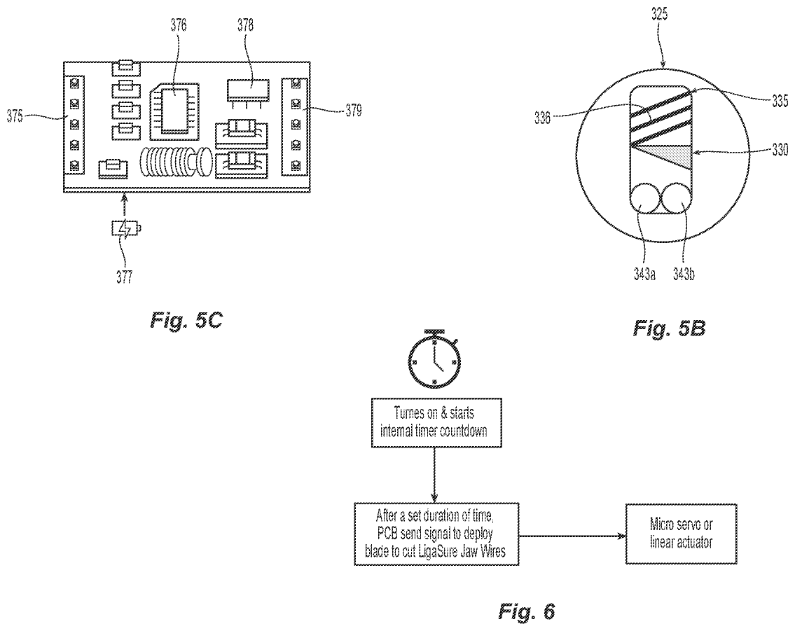

In another embodiment according to the present disclosure and similar to the embodiments described above, the first timer of the timing circuit 379 activates a clock to count down the overall use of the forceps 300 from initial usage and after a preset period of time, the timing circuit 379 electro-mechanically decouples or decommission the forceps 300 from the generator 141 thereby preventing reuse (FIG. 6). This added safety measure prevents reuse of the same disposable forceps 300.

More particularly, the forceps 300 includes a deactivating assembly 325 disposed within the housing 312 or the shaft 312 that electrically couples to the timing circuit 379. Upon expiration of the first timer, a signal is sent to the deactivating assembly 325 to mechanically disable or decommission the forceps 300 for reuse. Deactivating assembly 325 includes a deployment mechanism 335 operably associated with a deployable blade 330. Deployment mechanism 335 may include any combination of mechanical or electromechanical components, e.g., actuators and solenoids, that are configured to selectively deploy the blade 330 upon receiving a signal from the timing circuit 379. For example, upon the clock of the first timer running out, a signal may be sent to a solenoid which, in turn, activates or releases an actuator, e.g., spring 336, to deploy the blade 330 to cut one or both of the wire conductors 343a, 343b disposed within cable 343. As can be appreciated, this safety feature disables the disposable forceps 300 for subsequent use after a preset period of time has expired from initial use, i.e., the beginning of the surgery or initial use.

If forceps 300 is reusable, or reposable, the deactivating assembly 325 may be configured to flip an internal switch (not shown) or the like that can be reset after sterilization.

The various embodiments disclosed herein may also be configured to work with robotic surgical systems and what is commonly referred to as “Telesurgery.” Such systems employ various robotic elements to assist the surgeon and allow remote operation (or partial remote operation) of surgical instrumentation. Various robotic arms, gears, cams, pulleys, electric and mechanical motors, etc. may be employed for this purpose and may be designed with a robotic surgical system to assist the surgeon during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of surgeons or nurses may prep the patient for surgery and configure the robotic surgical system with one or more of the instruments disclosed herein while another surgeon (or group of surgeons) remotely control the instruments via the robotic surgical system. As can be appreciated, a highly skilled surgeon may perform multiple operations in multiple locations without leaving his/her remote console which can be both economically advantageous and a benefit to the patient or a series of patients.

The robotic arms of the surgical system are typically coupled to a pair of master handles by a controller. The handles can be moved by the surgeon to produce a corresponding movement of the working ends of any type of surgical instrument (e.g., end effectors, graspers, knifes, scissors, etc.) which may complement the use of one or more of the embodiments described herein. The movement of the master handles may be scaled so that the working ends have a corresponding movement that is different, smaller or larger, than the movement performed by the operating hands of the surgeon. The scale factor or gearing ratio may be adjustable so that the operator can control the resolution of the working ends of the surgical instrument(s).

The master handles may include various sensors to provide feedback to the surgeon relating to various tissue parameters or conditions, e.g., tissue resistance due to manipulation, cutting or otherwise treating, pressure by the instrument onto the tissue, tissue temperature, tissue impedance, etc. As can be appreciated, such sensors provide the surgeon with enhanced tactile feedback simulating actual operating conditions. The master handles may also include a variety of different actuators for delicate tissue manipulation or treatment further enhancing the surgeon's ability to mimic actual operating conditions.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as examples of particular embodiments. Those skilled in the art will envision other modifications within the scope and spirit of the claims appended hereto.

Although the foregoing disclosure has been described in some detail by way of illustration and example, for purposes of clarity or understanding, it will be obvious that certain changes and modifications may be practiced within the scope of the appended claims.

What is claimed is:

1. A surgical instrument, comprising:
a housing having a shaft extending therefrom;
an end effector assembly disposed at a distal end of the shaft, the end effector assembly including first and second jaw members movable between a first position wherein at least one of the jaw members is spaced relative to the other of the jaw members and a second position wherein the first and second jaw members cooperate to grasp tissue, at least one of the jaw members adapted to connect to an electrosurgical energy source;
a handle disposed on the housing and selectively moveable relative thereto to move the jaw members between the first and second positions;
a PCB disposed within the housing, the PCB including:
at least one accelerometer; and
a timing circuit including a first timer, the at least one accelerometer configured to activate the first timer of the timing circuit upon detecting movement of the surgical instrument after the surgical instrument is coupled to the electrosurgical energy source; and
a deactivating assembly disposed within the housing, the deactivating assembly operably associated with the timing circuit such that after expiration of the first timer, the deactivation assembly mechanically decommissions the surgical instrument for continued or subsequent use.

2. The surgical instrument according to claim 1, wherein the deactivating assembly mechanically decommissions the surgical instrument for continued use by severing internal electrical connections to the jaw members.

3. The surgical instrument according to claim 2, wherein the deactivating assembly includes a blade that selectively extends to sever at least one wire conductor connected to at least one jaw member upon expiration of the first timer.

4. The surgical instrument according to claim 3, wherein the deactivating assembly includes an actuator operably coupled to the blade, the actuator configured to selectively extend the blade upon expiration of the first timer.

5. The surgical instrument according to claim 4, wherein the actuator includes at least one of a spring or solenoid.

6. The surgical instrument according to claim 1, wherein the PCB includes a 3-axis accelerometer disposed thereon which is configured to detect handling of the surgical instrument.

7. The surgical instrument according to claim 1, wherein the PCB board includes vibration sensor configured to sense vibration of the surgical instrument during handling.

8. A surgical instrument, comprising:
a housing having a shaft extending therefrom for supporting an end effector assembly;
a PCB disposed within the housing, the PCB including:
at least one accelerometer; and
a timing circuit including a first timer, the at least one accelerometer configured to activate the first timer of the timing circuit upon detecting movement of the surgical instrument after the surgical instrument is coupled to an electrosurgical energy source; and
a deactivating assembly disposed within the housing, the deactivating assembly operably associated with the timing circuit such that after expiration of the first timer, the deactivation assembly mechanically decommissions the surgical instrument for continued or subsequent use.

9. The surgical instrument according to claim 2, wherein the deactivating assembly mechanically decommissions the surgical instrument for continued use by severing internal electrical connections to the end effector assembly.

10. The surgical instrument according to claim 9, wherein the deactivating assembly includes a blade that selectively extends to sever a wire conductor connected to the end effector assembly upon expiration of the first timer.

11. The surgical instrument according to claim 10, wherein the deactivating assembly includes an actuator operably coupled to the blade, the actuator configured to selectively extend the blade upon expiration of the first timer.

12. The surgical instrument according to claim 11, wherein the actuator includes at least one of a spring or solenoid.

13. The surgical instrument according to claim 8, wherein the PCB includes a 3-axis accelerometer disposed thereon which is configured to detect handling of the surgical instrument.

14. The surgical instrument according to claim 8, wherein the PCB board includes vibration sensor disposed thereon configured to sense vibration of the surgical instrument during handling.

15. A method of decommissioning an electrosurgical instrument, comprising:
coupling an electrosurgical instrument having an end effector assembly to an electrosurgical energy source;
using the electrosurgical instrument during a surgical procedure to induce an accelerometer to activate a first timer of a timer circuit of a PCB; and
mechanically deactivating the electrosurgical instrument after expiration of the first timer to decommission the electrosurgical instrument for continued or subsequent use.

16. The method of decommissioning an electrosurgical instrument according to claim 15, wherein the mechanically deactivating the electrosurgical instrument after expiration of the first timer includes severing internal electrical connections to the end effector assembly using a deactivating assembly.

17. The method of decommissioning an electrosurgical instrument according to claim 16, wherein the deactivating assembly includes a blade and an actuator operably coupled to the blade, the actuator configured to selectively extend the blade upon expiration of the first timer.

18. The method of decommissioning an electrosurgical instrument according to claim 15, wherein the accelerometer is a 3-axis accelerometer.

19. The method of decommissioning an electrosurgical instrument according to claim 15, further including initializing the electrosurgical instrument after coupling the electrosurgical instrument to the electrosurgical energy source.

20. The method of decommissioning an electrosurgical instrument according to claim 15, wherein the first timer is activated only after initialization of the electrosurgical instrument.
